(19) United States
(12) Patent Application Publication
(10) Pub. No.: US 2024/0269380 A1
(43) Pub. Date: Aug. 15, 2024

(54) SAFETY DEVICE FOR DRUG DELIVERY SYSTEM

(71) Applicant: AMGEN INC., Thousand Oaks, CA (US)

(72) Inventors: Jeffrey C. Yeary, Thousand Oaks, CA (US); Michael A. Johnson, Thousand Oaks, CA (US)

(21) Appl. No.: 18/569,735

(22) PCT Filed: Jun. 10, 2022
(86) PCT No.: PCT/US22/32963

§ 371 Date: Dec. 13, 2023

Related U.S. Application Data
(60) Provisional application No. 63/202,878, filed on Jun. 28, 2021.

(51) Int. Cl. A61M 5/148; A61M 39/04

(52) U.S. Cl. CPC A61M 5/148; A61M 39/04; A61M 2039/042

(57) ABSTRACT

A safety device for a drug delivery system includes a first member including a first arm having first and second ends and a length extending therebetween and a second member including a second arm having first and second ends and a length extending therebetween. The second end of the first member defines a first wall region. The first member further includes a first guard positioned at or near the second end thereof. The second member is rotatably coupled with the first member, and the second end thereof defines a second wall region and a needle guide member. The second member further includes a second guard positioned at or near the second end thereof. Upon rotatably positioning the second end of the first member near or adjacent to the second end of the second member, the first and second wall regions cooperate to define a container connector receptacle and the first and second guards cooperate to define a hand guard.

### CROSS-REFERENCE TO RELATED APPLICATION

Priority is claimed to U.S. Provisional Patent Application No. 63/202,878, filed Jun. 28, 2021, the entire contents of which are hereby incorporated by reference herein.

### FIELD OF DISCLOSURE

The present disclosure generally relates to drug delivery systems, and, more particularly, to safety devices for intravenous (IV) drug delivery systems.

### BACKGROUND

Drugs are administered to treat a variety of conditions and diseases. Intravenous (“IV”) therapy is a drug dosing process that delivers drugs directly into a patient's vein using an infusion contained in a delivery container such as IV bag, a glass vial, and/or other pliable bag or container, and tubing connected to a needle subsystem that fluidically communicates with the reservoir through the pump assembly collectively called infusion set. These drug dosings may be performed in a healthcare facility, or in some instances, at remote locations such as a patient's home. In some examples, these containers may be initially prefilled with a fluid (e.g., a saline solution) and may require additional substances such as, for example, drug products or other solutions, to be added there to prior to administration. Oftentimes, needle syringes are used to insert the additional substances into the container by piercing a seal or septum. Occasionally, a user may inadvertently stick themselves with the syringe needle when attempting to fill the container. Similarly, a user may inadvertently puncture the container during the filling process, thus losing its contents and wasting material.

As described in more detail below, the present disclosure sets forth systems and methods for safety devices for drug delivery systems embodying advantageous alternatives to existing systems and methods, and that may address one or more of the challenges or needs mentioned herein, as well as provide other benefits and advantages.

### SUMMARY

In accordance with a first aspect, a safety device for a drug delivery system includes a first member including a first arm having first and second ends and a length extending therebetween and a second member including a second arm having first and second ends and a length extending therebetween. The second end of the first member defines a first wall region. The first member further includes a first guard positioned at or near the second end thereof. The second member is rotatably coupled with the first member, and the second end thereof defines a second wall region and a needle guide member. The second member further includes a second guard positioned at or near the second end thereof. Upon rotatably positioning the second end of the first member near or adjacent to the second end of the second member, the first and second wall regions cooperate to define a container connector receptacle and the first and second guards cooperate to define a hand guard.

In some examples, the needle guide member may include a body defining an opening therethrough that is dimensioned to slidably receive and align a needle. In some of these forms, the needle guide member may include a first side having a tapered guiding region. In some approaches, at least one of the first wall region or the second wall region may include at least one locking ledge.

Further, in some approaches, the first end of the first member is pivotable relative to the first end of the second member. In these and other examples, the first end of the first member includes a throughbore, and the first end of the second member includes a throughbore. The safety device may further include a rod member that is insertable through the throughbore of the first member and the throughbore of the second member.

In some forms, the hand guard may be in the form of a disk extending in a radial direction. At least one of the first guard or the second guard may include a notched region positioned along an outer periphery thereof to accommodate at least a portion of a second container connector.

In some examples, the safety device may be constructed from a plastic material.

In accordance with a second aspect, a drug delivery system include a prefilled delivery container, a container connector, and a safety device. The prefilled delivery container includes a container body defining an interior cavity and at least one opening to permit access to the interior cavity. The container connector is operably coupled with the at least one opening to selectively restrict access to the interior cavity. The safety device includes a first member including a first arm and a second member including a second arm. The first arm has a first end, a second end, and a length extending therebetween. The second end defines a first wall region. The first member further includes a first guard positioned at or near the second end of the first arm. The second member is rotatably coupled with the first member. The second arm has a first end, a second end, and a length extending therebetween. The second end of the second member further defines a second wall region, a second guard positioned at or near the second end of the second arm, and a needle guide member positioned at or near the second end of the second arm. The first wall region of the first member and the second wall region of the second member cooperate to define a container connector receptacle that accommodates at least a portion of the container connector, and the first guard of the first member and the second guard of the second member cooperate to define a hand guard.

### BRIEF DESCRIPTION OF THE DRAWINGS

The above needs are at least partially met through provision of the systems and approaches for safety devices for drug delivery systems described in the following detailed description, particularly when studied in conjunction with the drawings, wherein:

FIG. 1 illustrates an upper side perspective view of an example safety device for use with an example drug delivery system in accordance with various embodiments;

FIG. 2 illustrates an upper side perspective view of the example safety device of FIG. 1 in accordance with various embodiments;

FIG. 3 illustrates a lower side perspective view of the example safety device of FIGS. 1 & 2 in accordance with various embodiments;

FIG. 4 illustrates a lower side perspective view of the example safety device of FIGS. 1-3 in accordance with various embodiments;

FIG. 5 illustrates a bottom perspective view of the example safety device of FIGS. 1-4 in accordance with various embodiments;

FIG. 6 illustrates a side perspective view of a first member of the example safety device of FIGS. 1-5 in accordance with various embodiments;

FIG. 7 illustrates a side elevation view of the first member of the example safety device of FIGS. 1-6 in accordance with various embodiments;

FIG. 8 illustrates a side perspective view of a second member of the example safety device of FIGS. 1-7 in accordance with various embodiments;

FIG. 9 illustrates a side elevation view of the second member of the example safety device of FIGS. 1-8 in accordance with various embodiments;

FIG. 10 illustrates a side elevation cross sectional view of the second member of the example safety device of FIGS. 1-8 in accordance with various embodiments;

FIG. 11 illustrates the example safety device of FIGS. 1-10 coupled with an example delivery container and grasped by a user in accordance with various embodiments;

FIG. 12 illustrates the first member of the example safety device of FIGS. 1-11 coupled with an example container having an example container connector in accordance with various embodiments;

FIG. 13 illustrates an example needle coupled with the example safety device of FIGS. 1-12 in accordance with various embodiments; and

FIG. 14 illustrates the example safety device of FIGS. 1-13 coupled with the example container and the example needle in accordance with various embodiments.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, a safety device is provided for use with drug delivery containers. In some examples, the safety device is in the form of a shield that is positionable about a container connector operably coupled with a drug delivery container to shield a portion of the drug delivery container from a needle syringe. In such examples, the safety device may include a guiding member to assist with proper alignment of the needle prior to insertion into the container connector (and thus, the drug delivery container).

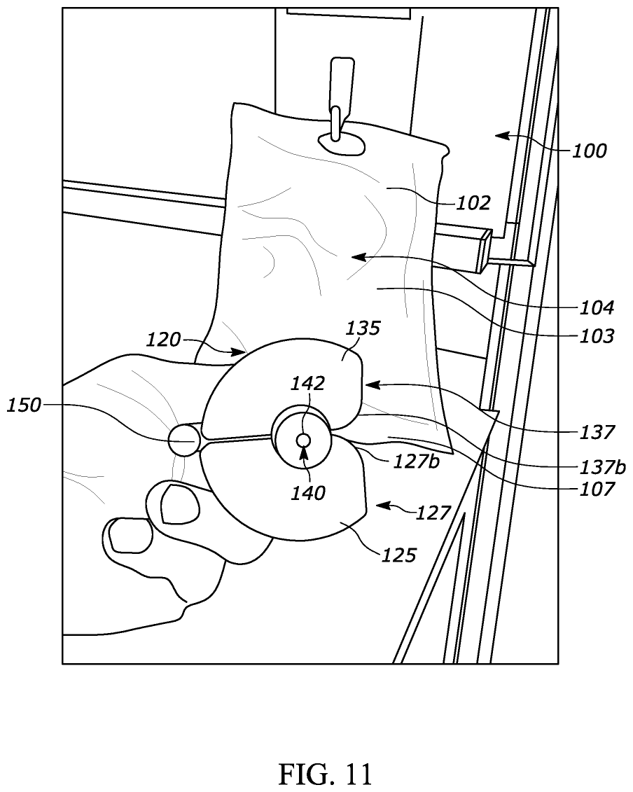

Turning to the Figures, a safety device 120 is provided for use with a drug delivery system 100 (FIGS. 11-14). Generally, and as illustrated in FIGS. 11-14, the drug delivery system 100 includes a prefilled delivery container 102, a needle syringe 110, and the safety device 120. The prefilled delivery container 102 includes a container body 103 defining an inner volume 104, an opening 105, a first delivery container connector or adapter 106, and a second delivery container connector or adapter 107. In some examples, the prefilled delivery container 102 is in the form of an IV drip bag constructed from a plastic or other material, e.g., 250 mL 0.9% Sodium Chloride IV bag constructed of a suitable material such as polyolefin, non-DEHP (diethylhexl phthalate), PVC, polyurethane, or EVA (ethylene vinyl acetate) and can be filled to a volume of approximately 270 mL to account for potential moisture loss over long-term storage. Other examples of suitable delivery containers are possible such as, for example, a glass bottle or container. Example suitable prefilled delivery containers 102 are described in U.S. Appln. No. 62/804,447, filed on Feb. 12, 2019, the contents of which are incorporated by reference in their entirety.

The prefilled delivery container 102 contains a predetermined quantity (e.g., a volume) of excipient solution. For example, the prefilled delivery container 102 can include a predetermined quantity of a saline solution (e.g., between approximately 25 mL and 500 mL of 0.9% Sodium Chloride per dose, and preferably, approximately 110 mL or approximately 270 mL per dose, depending on the size of the container) and a predetermined quantity of an IV stabilizing solution (“IVSS”). In some examples, the IVSS and/or the saline solution may be provided as a percentage or ratio of an overall volume of solution. In these examples, suitable quantities of IVSS may range between approximately 2% and approximately 15% (e.g., between approximately 1 mL in a 50 mL container 102 and approximately 25 mL in a larger, 270 mL container per dose). In some examples, the prefilled delivery container 102 may have a total volume of approximately 270 mL

The first delivery container connector or adapter 106 is in the form of a septum or a seal that is operably coupled with the opening 105 to seal the opening 105 and/or to retain contents disposed within the inner volume 104 of the prefilled delivery container 102. The first delivery container adapter 106 may be a closed system transfer device (“CSTD”) that allows for transfer of the drug and/or fluids into the container body 103. Example CSTD devices may include the OnGuard CSTD provided by B. Braun Medical Inc., BD PhaSeal CSTD components, Equashield CSTD, Codon CSTD, and the like. Further, non-closed system transfer devices may be used such as West Pharmaceuticals vial and bag adapters. Other examples are possible. The prefilled delivery container 102 may include any number of delivery container adapters 106 having different specifications (e.g., port sizes) to accommodate the use of different needle syringes 110 or other drug containers. The second delivery container connector 10 is in the form of an IV line outlet that allows tubing to be coupled thereto in order to deliver the prescribed drug. It is appreciated that the container 102 is just one example of a suitable drug delivery device that may be used with the safety device 120.

The safety device 120 is in the form of a shield that includes a first member 121 and a second member 131. The first member 121 includes a first arm 122 having a first end 122a, a second end 122b, and a length 122c extending therebetween. The second end 122b of the first arm 122 defines a first wall region 123. The first member 121 further includes a first guard 125 positioned at or near the second end 122b. As illustrated in the Figures, the first arm 122 is formed integrally with the first guard 125, however in other examples, the first arm 122 and the first guard 125 may be discrete components operably coupled with each other via any number of suitable approaches.

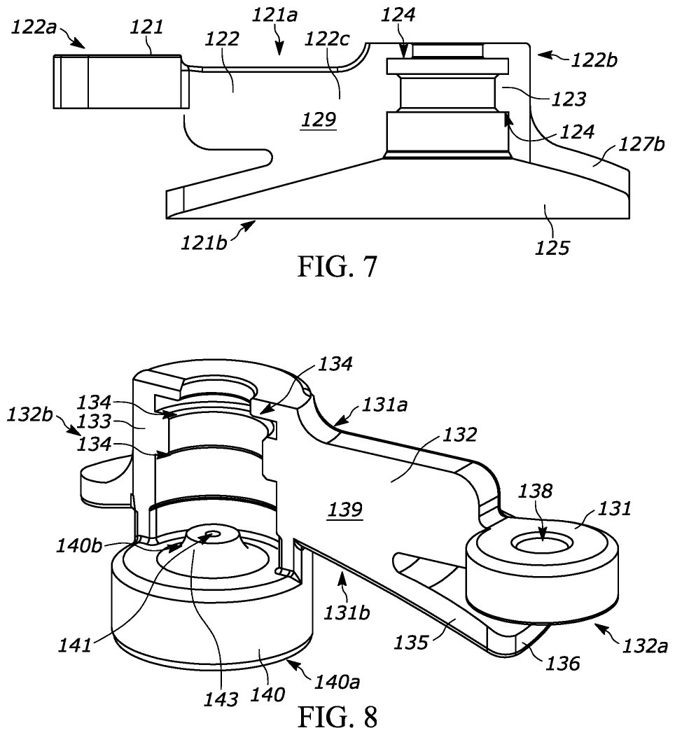

With particular reference to FIGS. 6 and 7, the first wall region 123 is in the form of a cavity formed in the second end 122b of the first arm 122. More specifically, the first wall region 123 is approximately semi-cylindrical and extends between an upper side 121a of the first member 121 to a lower side 121b thereof. The first wall region 123 includes any number of ledges 124 formed thereon which are generally dimensioned to correspond to at least a portion of the shape of the first container connector 106.

The first guard 125 is in the form of a disk or a shield that extends radially outwardly from the second end 122b of the first arm 122. The first guard 125 has a generally tapered arrangement and includes a curved outer wall 126. The outer wall 126 includes a transition region 127 that includes a generally linear portion 127a and a curved or notched portion 127b.

Like the first member 121, the second member 131 includes a second arm 132 having a first end 132a, a second end 132b, and a length 132c extending therebetween. The second end 132b of the second arm 132 defines a second wall region 133. The second member 131 further includes a second guard 135 positioned at or near the second end 132b and a needle guide member 140. As illustrated in the Figures, the second arm 132 is formed integrally with the second guard 135, however in other examples, the second arm 132 and the second guard 135 may be discrete components operably coupled with each other via any number of suitable approaches.

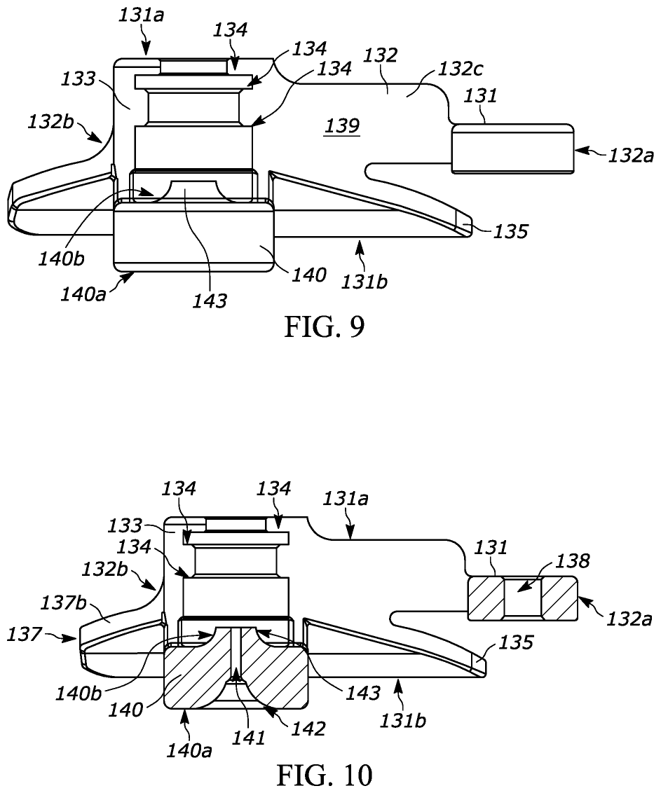

With particular reference to FIGS. 8-10, the second wall region 133 is in the form of a cavity formed in the second end 132b of the second arm 132. More specifically, the second wall region 133 is approximately semi-cylindrical and extends between an upper side 131a of the second member 131 to a lower side 131b thereof. The second wall region 133 includes any number of ledges 134 formed thereon which are generally dimensioned to correspond to at least a portion of the shape of the first container connector 106.

The second guard 135 is in the form of a disk or a shield that extends radially outwardly from the second end 132b of the second arm 132. The second guard 135 has a generally tapered arrangement and includes a curved outer wall 136. The outer wall 136 includes a transition region 137 that includes a generally linear portion 137a and a curved or notched portion 137b.

The needle guide member 140 is also positioned at or near the second end 132b of the second arm 132. More specifically, the needle guide member 140 is positioned on the lower side 131b of the second member 131 and is generally below the second guard 135. The needle guide member 140 is in the form of a generally cylindrical body that defines an opening 141 therethrough. Other examples of suitable shapes and/or arrangements are possible. As illustrated in FIG. 10, the needle guide member 140 includes a first side 140a and a second side 140b. The first side 140a of the needle guide member 140 includes a generally tapered or conical surface 142 extending towards the second side 140b. Further, the second side 140b of the needle guide member 140 includes a generally tapered or conical protrusion 143 extending towards and/or into the second wall region 133. Other arrangements are possible.

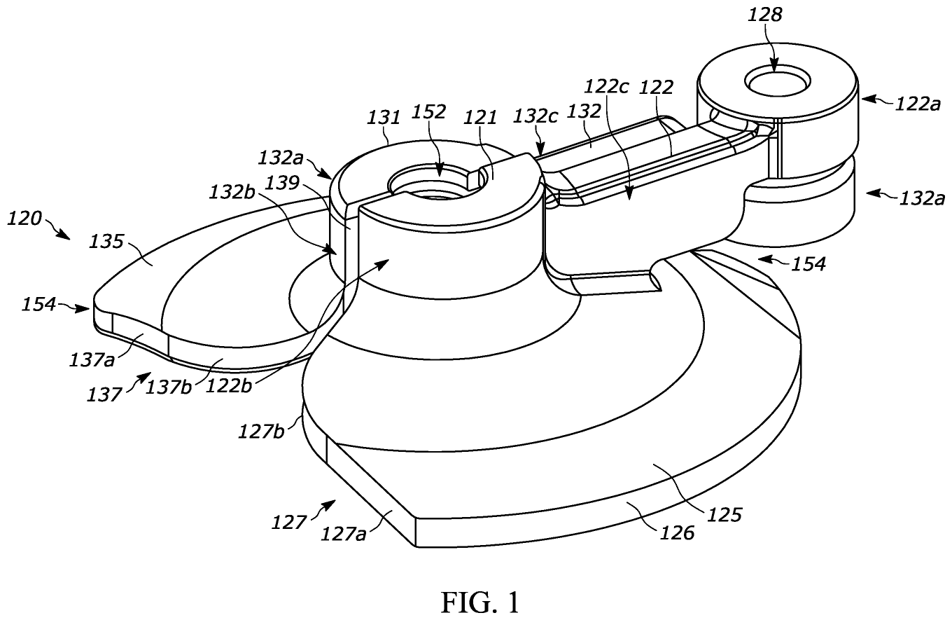

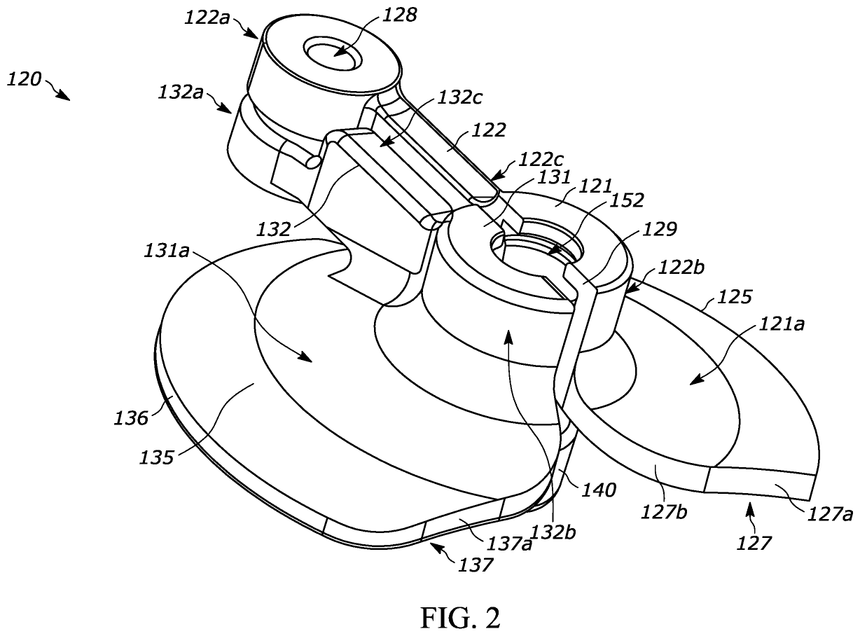

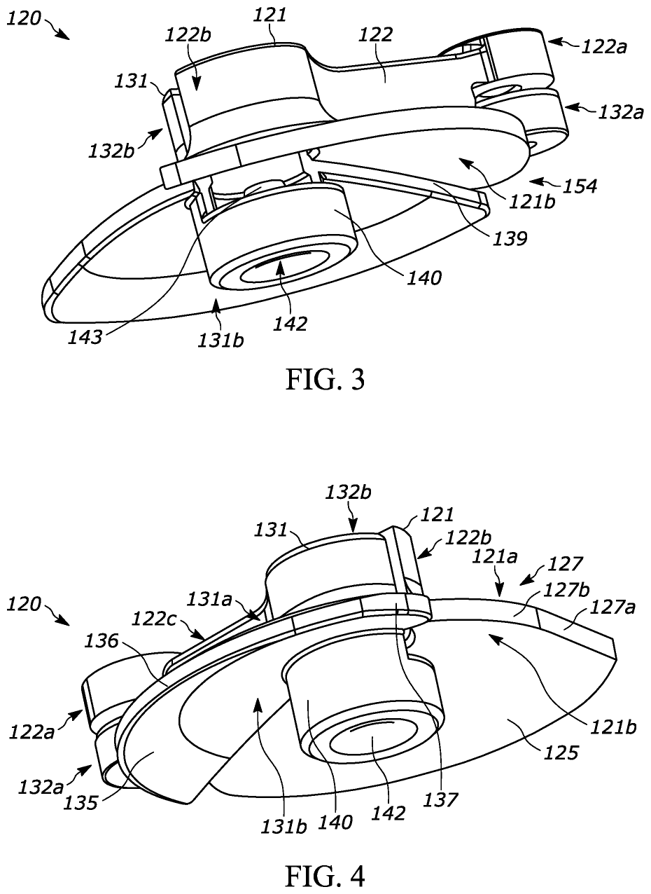

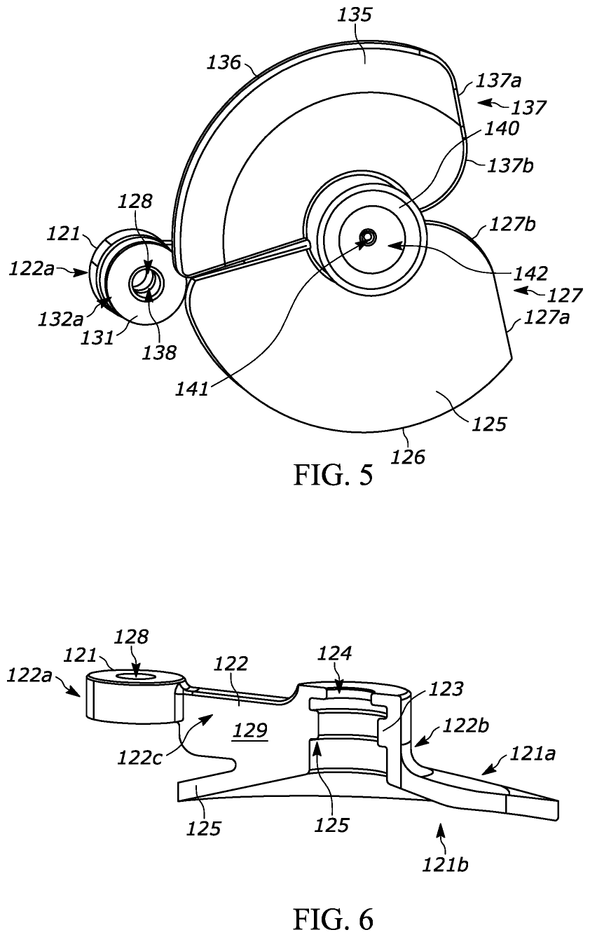

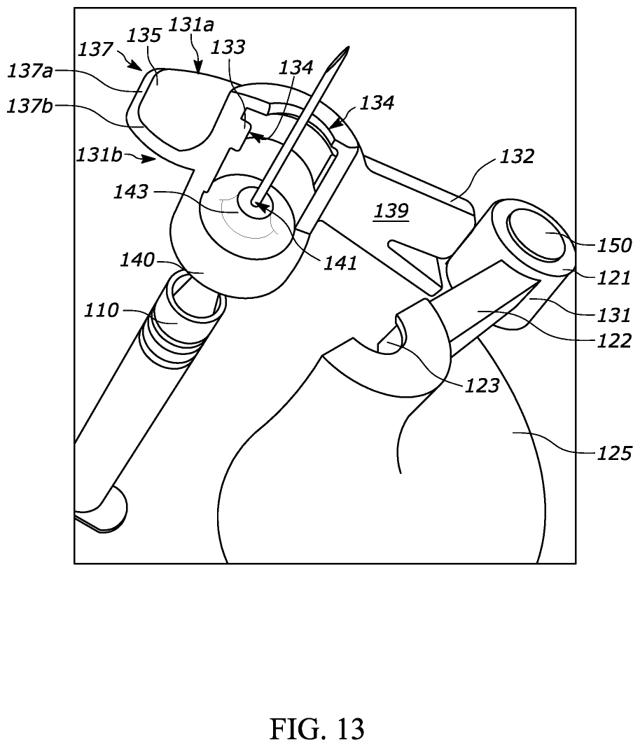

The first end 122a of the first arm 122 includes a throughbore 128 formed therethrough. Further, the first arm 122 includes a facing surface 129. The first end 132a of the second arm 132 similarly includes a throughbore 138 formed therethrough. The first arm additionally includes a facing surface 139. The first member 121 and the second member 131 are adapted to be rotatably coupled with each other to form the safety device 120. More specifically, upon aligning the first ends 122a, 132a of the first and second arms 122, 132, a pin 150 (FIGS. 11, 13, & 14) is insertable into the throughbores 128, 138 of the respective first and second arms 122, 133. As such, the first and second arms 122, 132 may rotate relative to each other between an opened configuration (FIG. 13) and a closed configuration (FIGS. 1-5, 11, & 14). When the first and second arms 122, 132 are positioned in the closed configuration, the respective facing surfaces 129, 139 may abut or be in proximity to each other. In such a closed configuration, the safety device 120 forms a complete shield member. More specifically, in this configuration, the first and second wall regions 123, 133 cooperate to define a container connector receptacle 152 which receives the first container connector 106. Further, in this configuration, the first guard 125 and the second guard 135 cooperate to define a hand guard 154.

The safety device 120 may be constructed from any number of suitable materials such as, for example a silicone material or other polymeric material, a metal, or any number of additional suitable materials. All or any portions of the safety device 120 may be flexible, semi-rigid, or rigid. In some examples, the safety device 120 may be produced via additive manufacturing techniques. Other examples are possible.

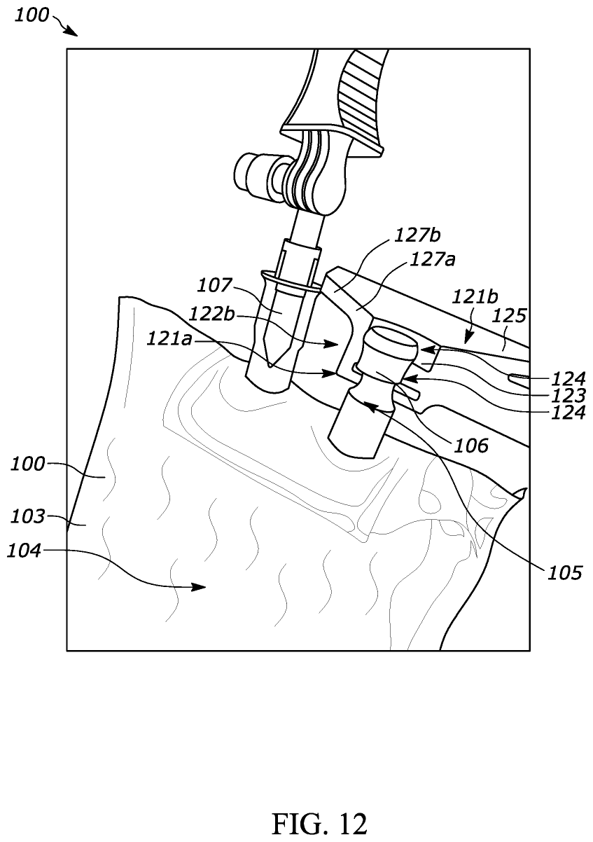

In use, and as illustrated in FIG. 12, a user may position the first delivery container connector 106 within one of the first or second wall regions 123, 133 of the respective first or second member 122, 132. As illustrated in FIG. 12, the ledges 124 of the first wall region 123 are positioned adjacent to corresponding flanges or surfaces formed on the first delivery container connector 106. Further, in some examples, the curved or notched portion 127b of the outer wall 126 of the first guard 125 may be positioned adjacent to the second delivery container connector 107.

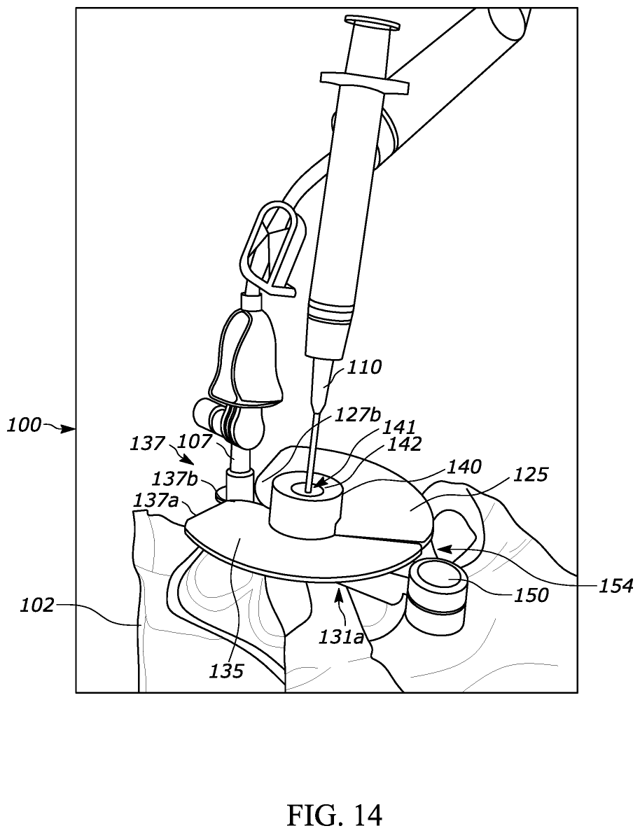

With particular reference to FIG. 11, a user may grasp the first and second arms 122, 132 and urge them towards each other such that the facing surfaces 129, 139 are adjacent to and/or abut each other. In this configuration, a portion of the user's hand, the first container connector 106, and a portion of the delivery container 102 are shielded by the formed hand guard 154 (i.e., the first and the second guards 125, 135). In this closed configuration, the needle guide member 140 becomes aligned with the formed container connector receptacle 152. Further, and as previously noted, at least a portion of the first container connector 106 is disposed within the first container connector receptacle 152 and is at least partially restricted from moving in an axial direction that is parallel to the opening 141 of the needle guide member 140. In some examples, in this closed configuration, the generally tapered or conical protrusion 143 formed on the second side 140b of the needle guide member 140 may abut a portion of the first container connector 106. Additionally, and as illustrated in FIG. 14, at least a portion of the second container connector 107 may be disposed within the curved or notched portions 127b, 137b formed by the first and second guards 125, 135 for increased stability.

As illustrated in FIGS. 13 & 14, the needle syringe 110 is insertable into the opening 141 of the needle guide member 140. The needle syringe 110 may be moved along the tapered or conical surface 142 and through the opening 141. Upon continued urging of the needle syringe 110 into the opening 141, the needle syringe 110 may pierce the first container connector 106 to permit additional material (e.g., drug product, fluids, etc.) to be inserted into the inner volume 104 of the delivery container 102 or to be drawn material therefrom.

So configured, the system described herein ensures the needle of the needle syringe 110 is safely inserted through the septum and/or connector and into the delivery container. The safety device 120 is universal and can be used with connectors having a variety of different sizes, shapes, and/or configurations. The safety device 120 ensures a user's hands are not poked, thereby allowing the safe insertion of the needle while protecting the user's hands and fingers. Such a device may be efficiently used by clinicians, pharmacists, other healthcare professionals that dose delivery containers for patients. The system advantageously shields portions of the delivery container from the needle, thereby reducing inadvertent punctures of the container that otherwise may require the contents of the container to be disposed of prior to use.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa). Aranesp® (darbepoetin alfa), Dynepo® (epoetin delta), Mircera® (methyoxy polyethylene glycol-epoetin beta), Hematide@, MRK-2578, INS-22, Retacrit® (epoetin zeta), Neorecormon@ (epoetin beta), Silapo@ (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-a4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin@ (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-a4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to lgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACl-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-a531 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGβ mAb (MDX-1307); anti-mesothelin dsFv-PE38 conjugate (CAT-5001); anti-PD1mAb (MDX-1106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/FIt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of lgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human lgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human lgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19 x CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3 x epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A safety device for a drug delivery system, the safety device comprising:
a first member including a first arm having a first end, a second end, and a length extending therebetween, the second end of the first member defining a first wall region, the first member further including a first guard positioned at or near the second end of the first arm;
a second member adapted to be rotatably coupled with the first member, the second member including a second arm having a first end, a second end, and a length extending therebetween, the second end of the second member defining a second wall region, a second guard positioned at or near the second end of the second arm, and a needle guide member positioned at or near the second end of the second arm;
wherein upon rotatably positioning the second end of the first member near or adjacent to the second end of the second member, the first wall region of the first member and the second wall region of the second member cooperate to define a container connector receptacle, and the first guard of the first member and the second guard of the second member cooperate to define a hand guard.

2. The safety device of claim 1, wherein the needle guide member includes a body defining an opening therethrough dimensioned to slidably receive and align a needle.

3. The safety device of claim 2, wherein the needle guide member includes a first side having a tapered guiding region.

4. The safety device of claim 1, wherein at least one of the first wall region or the second wall region includes at least one locking ledge.

5. The safety device of claim 1, wherein the first end of the first member is adapted to pivot relative to the first end of the second member.

6. The safety device of claim 5, wherein the first end of the first member includes a throughbore and the first end of the second member includes a throughbore, the safety device further comprising a rod member adapted to be inserted through the through bore of the first member and the throughbore of the second member.

7. The safety device of claim 1, wherein the hand guard comprises a disk extending in a radial direction.

8. The safety device of claim 1, wherein at least one of the first guard or the second guard includes a notched region positioned along an outer periphery thereof to accommodate at least a portion of a second container connector.

9. The safety device of claim 1, wherein the safety device is constructed from a plastic material.

10. A drug delivery system including:
a prefilled delivery container including a container body defining an interior cavity and at least one opening to permit access to the interior cavity;
a container connector operably coupled with the at least one opening to selectively restrict access to the interior cavity; and
a safety device including:
a first member including a first arm having a first end, a second end, and a length extending therebetween, the second end of the first member defining a first wall region, the first member further including a first guard positioned at or near the second end of the first arm;
a second member adapted to be rotatably coupled with the first member, the second member including a second arm having a first end, a second end, and a length extending therebetween, the second end of the second member defining a second wall region, a second guard positioned at or near the second end of the second arm, and a needle guide member positioned at or near the second end of the second arm;
wherein the first wall region of the first member and the second wall region of the second member cooperate to define a container connector receptacle adapted to accommodate at least a portion of the container connector, and the first guard of the first member and the second guard of the second member cooperate to define a hand guard.

11. The drug delivery system of claim 10, wherein the needle guide member includes a body defining an opening therethrough dimensioned to slidably receive and align a needle.

12. The drug delivery system of claim 11, wherein the needle guide member includes a first side having a tapered guiding region.

13. The drug delivery system of claim 10, wherein at least one of the first wall region or the second wall region includes at least one locking ledge adapted to retain and permit axial movement of at least a portion of the container connector.

14. The drug delivery system of claim 10, wherein the first end of the first member is adapted to pivot relative to the first end of the second member includes a throughbore.

15. The drug delivery system of claim 14, wherein the first end of the first member includes a throughbore and the first end of the second member includes a throughbore, the safety device further comprising a rod member adapted to be inserted through the through bore of the first member and the throughbore of the second member.

16. The drug delivery system of claim 10, wherein the hand guard comprises a disk extending in a radial direction.

17. The drug delivery system of claim 10, wherein the prefilled delivery container further includes a second container connecter, wherein at least one of the first guard or the second guard includes a notched region positioned along an outer periphery thereof to accommodate at least a portion of a second container connector.

18. The drug delivery system of claim 10, wherein the safety device is constructed from a plastic material.
